(19) United States
(12) Patent Application Publication
(10) Pub. No.: US 2025/0332373 A1
(43) Pub. Date: Oct. 30, 2025

(54) Modeling Compound Sensory Play System

(71) Applicant: HASBRO, INC., Pawtucket, RI (US)

(72) Inventors: Andrea Renee Ehret, Cranston, RI (US); Douglas Drew Papuga, Pawtucket, RI (US)

(21) Appl. No.: 19/190,394

(22) Filed: Apr. 25, 2025

Related U.S. Application Data
(60) Provisional application No. 63/639,278, filed on Apr. 26, 2024.

(51) Int. Cl. A61M 21/00; A63H 33/00; A63H 33/08

(52) U.S. Cl. CPC A61M 21/00; A63H 33/001; A63H 33/088; A61M 2021/0044; A61M 2205/59

(57) ABSTRACT

A sensory play system can include one or more stackers, each stacker comprises two walls connected at a hinge and designed to be moved between open and closed positions, one or both of the two walls comprising a transparent panel, wherein in the closed position an interior region is defined between the two walls; and a modeling compound to be received in the interior region.

### CROSS-REFERENCE TO RELATED APPLICATION

The benefit of priority to U.S. Provisional Patent Application No. 63/639,278 filed Apr. 26, 2024, and the disclosure is incorporated herein by reference in its entirety.

### FIELD

The disclosure relates a sensory play system using a modeling compound.

### BACKGROUND

Sensory play stimulates multiple senses providing children with an interactive play system to support learning and development of they're senses. Benefits of sensory play can include the development of fine motor skills, brain and memory development, and encouragement of creativity.

### BRIEF DESCRIPTION OF THE DRAWINGS

FIG. 1 is an image of a stacker of a sensory play system in accordance with the disclosure, the stacker being shown in the open position.

FIG. 2 is an image of the stacker in the closed position.

FIG. 3 is an image of the stacker in the open position showing modeling compound contained in the interior region.

FIG. 4 is an image of the stacker in the closed position showing modeling compound contained in the interior region.

FIG. 5 is an image of two stackers connected at the connection pins.

FIG. 6 is an image of a system in accordance with the disclosure.

FIG. 7 is an image of a system in accordance with the disclosure.

FIG. 8 shows a play pattern for a system in accordance with the disclosure.

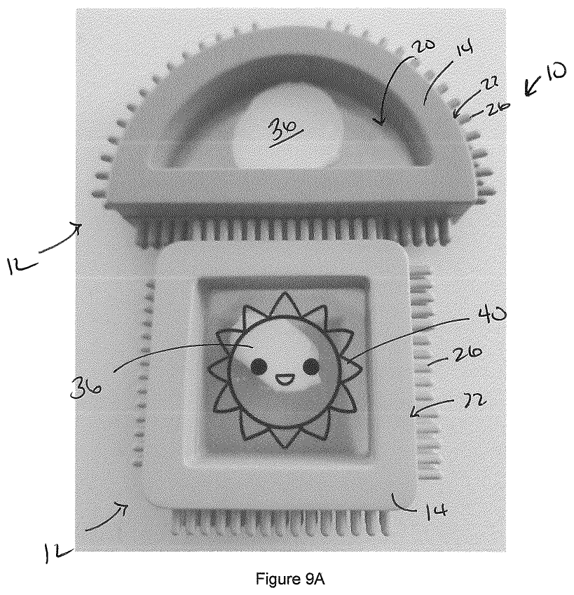

FIGS. 9A and 9B are images of a system having images arranged on the transparent panela and showing the system in use with a modeling compound disposed in the interior region of the stacker.

### DETAILED DESCRIPTION

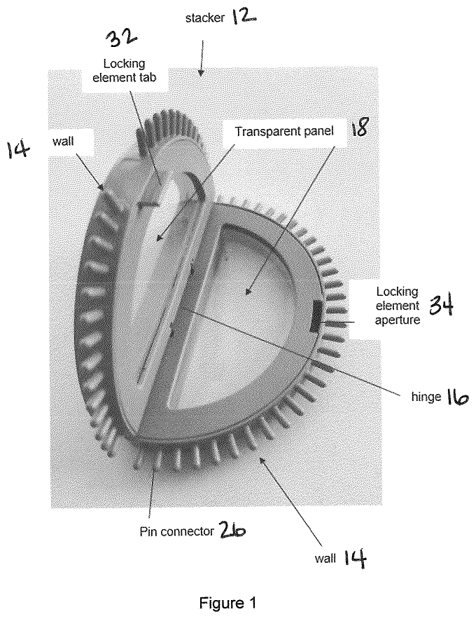

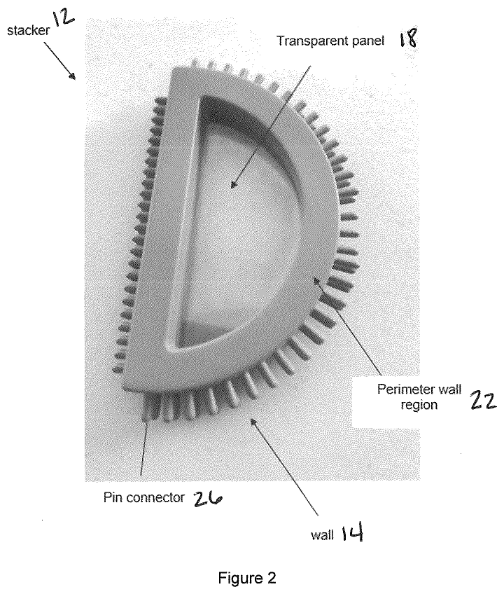

The sensory play system 10 in accordance with the disclosure can include one or more stackers 12, each stacker includes first and second hinged walls 14 that are movable between open and closed positions about a living hinge 16 as shown in FIGS. 1 and 2. At least one wall 14 includes a transparent, flexible panel. In embodiments, both walls 14 can include transparent panels 18. The stackers 12 can each include an interior region defined between the transparent panels 18. A composition, such as a modeling compound, a gel compound, or other sensory compound, can be placed in the interior region 20 and be engaged with through the transparent panels 18. For example, a model compound can be pressed upon and changed in shape or moved within the interior region 20 through interaction with the transparent panels 18 and without having to directly contact the modeling compound itself. This can be advantageous for younger users, where interaction with the modeling compound itself is not desired for a variety of reasons, including to improve safety, such as preventing the compound from being ingested, and/or ease of clean up.

A sensory play system 10 in accordance with the disclosure can include two or more stackers 12, the two or more stackers 12 being designed for interaction or engagement using one or more pin structures as described in detail below to provide a play pattern that includes a building element in addition to or as an alternative to engagement with a compound placed in the interior region 20.

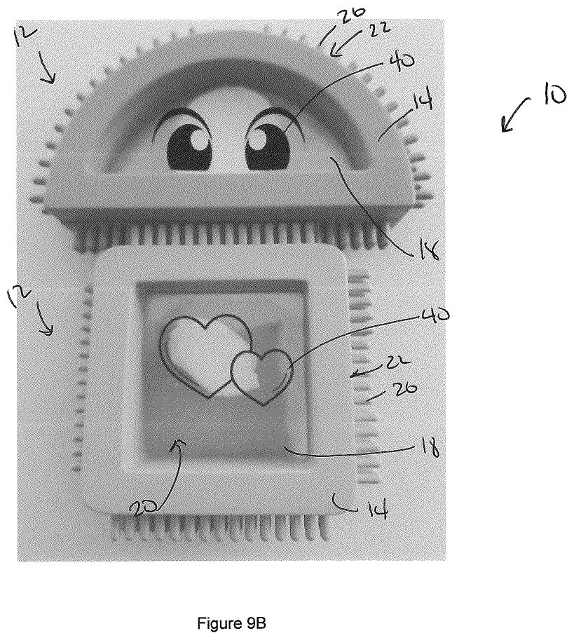

The first and second walls 14 cooperate to define an interior region 20. The interior region 20 can be defined between the two transparent flexible panels, for example. In embodiments, only one wall 14can include a transparent panel and the interior region 20 can be arranged between the transparent panel and a nontransparent panel region of the opposed wall. For example, the nontransparent panel region on the opposed wall 14can be colored, include an image, and/or can include a texture or texture features against which the modeling compound can be pressed. For example, the nontransparent panel can include an image that is visible through the transparent panel and against which the modeling compound can be disposed allowing the user to interact with or enhance the image with the modeling compound. Referring to FIG. 9, for example, an image can be provided on the transparent panel and the user can arrange a modeling compound within the interior region 20, positioned relative to the image to provide a color effect to the image when viewed through the transparent panel. The image can be any desired image, such as a shape, a face, elements of a face (e.g., eyes), a flower, pattern, or the like.

The texture and/or texture feature (not shown) can be raised portions and/or recesses formed in the non-transparent panel. The texture and/or texture feature can be formed into a transparent panel in some embodiments. The texture and/or texture features can be a pattern such that when the modeling compound is pressed against the texture and/or texture features, a pattern is imparted into the modeling compound. For example, the modeling compound can be inserted into the interior region 20 and then the walls 14 can be closed. The modeling compound can then be pressed or otherwise engaged through the transparent panel against the texture and/or textured features. A pattern in the texture and/or texture features can reveal an image or other pattern in the modeling compound after pressing against the features. This provides a tactile sensory experience in both interaction with the modeling compound as well as with the textured and/or raised features. Any known textures and means of providing texture on a surface can be used herein.

The stackers 12 can further include perimeter wall regions disposed about the transparent panels 18. The perimeter wall regions can be, for example, a rigid plastic material, and the transparent panels 18 can be a more flexible plastic material. For example, the transparent panels 18 can be formed of PET.

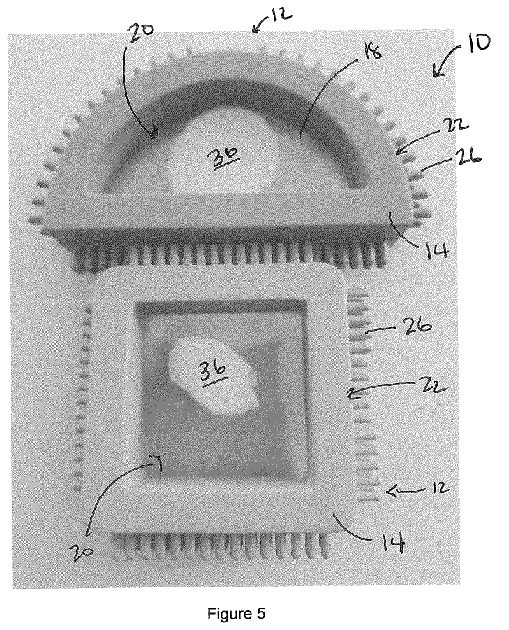

Referring to FIG. 5, the perimeter wall regions can be provided with one or more connecting pins 26 that allows a stacker 12 to be connected to another stacker 12 through engagement of the pins 26. Alternatively or additionally, the perimeter wall regions can be provided with one or more pins 26 and/or one or more apertures for receiving the pins 26. The stackers 12 can then interact for connection through engagement of a one or more of the pins 26 with one or more of the apertures. This can provide a play system 10 in which the stackers 12 can be used as both a building system 10 and for interaction with the modeling compound contained within the interior region 20 of the stacker.

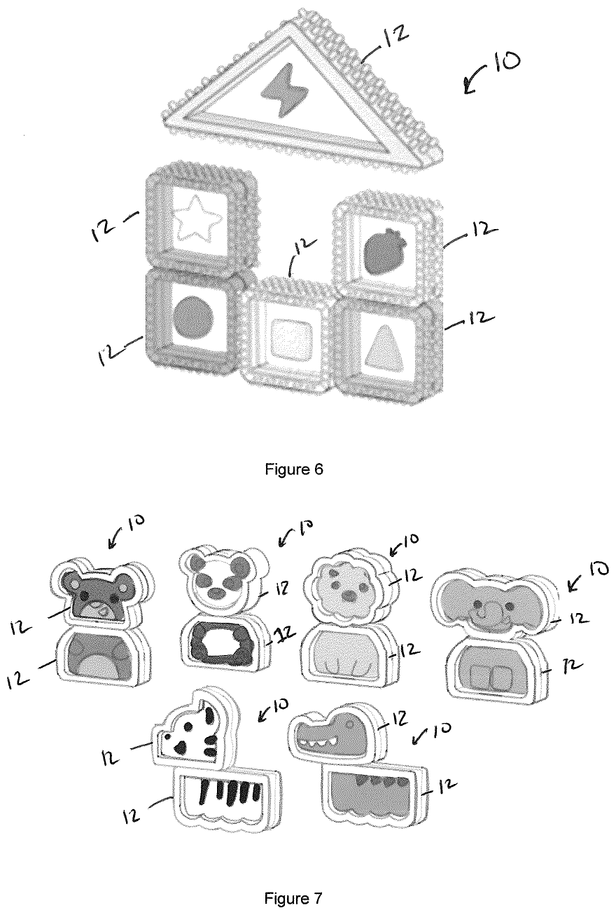

For example, referring to FIG. 1, the stacker 10 can have a single row of pins arranged around the perimeter edge of one or more walls. Referring to FIG. 6, the perimeter can include one or more rows of pins 26 extending outwardly from the wall 15. For example, the spacing between pins 26 can be sized such that a pin from another stacker 12 could fit within the spacing, the spacing thereby serving as an aperture for the pin 26 and allowing connection of the two stackers 12. The pins can be arranged in any desired configuration, on any number of the edges or perimeter surface of the stacker, and provided in any suitable number or amount. For example, a single row of pins 26 can be provided on one or more of the perimeter edges of one or more walls. For example, two or more rows of pins 26 can be provided on one or more of the perimeter edges of one or more of the walls. For example, each of the perimeter edges can be provided with one or more pins 26 and/or one or more rows of pins 26.

The stacker 12 can include a locking element 30 that retains the walls 14 in the closed position during interaction with the modeling compound 36 through the transparent panels 18. The locking element 30 can include a locking element tab 32 provided on one wall 14that is received in a locking element aperture 34 provided on the other wall 14. The tab 32 and the aperture 34 can cooperate to lock the two walls 14 in the closed position until sufficient force is applied to disengage the tab from the aperture. Any suitable locking mechanisms can be used to retain the walls 14 in the closed position during play or interaction of the modeling compound 36 through the transparent panels 18. The locking element 30 can be provided as a child-safe locking mechanism to prevent a child from opening the stacker.

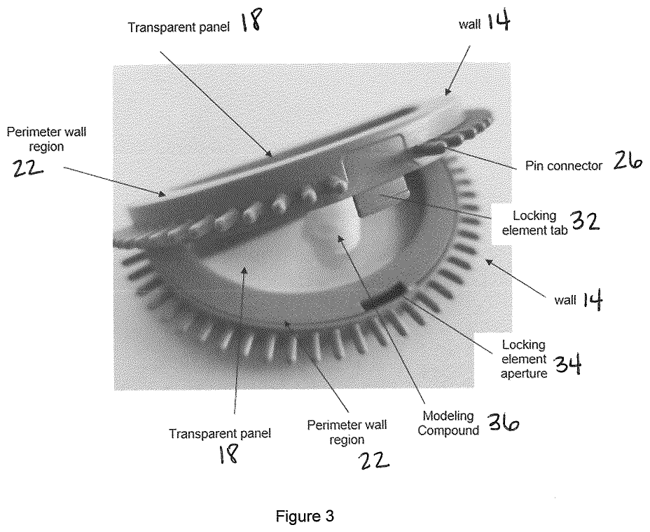

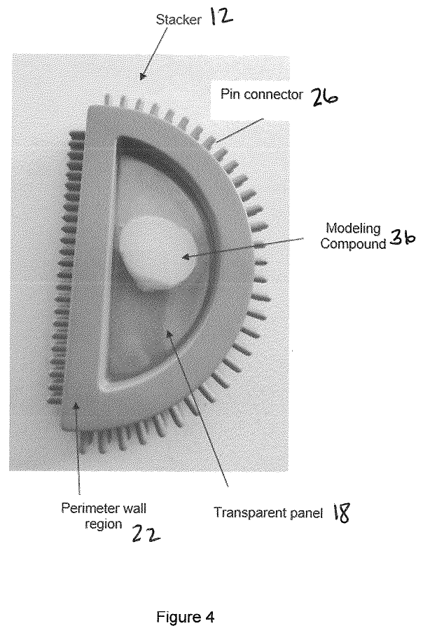

Referring to FIGS. 3 and 4, the interior region 20 is capable of receiving a modeling compound 36. When closed, a user can interact with the modeling compound 36 disposed in the interior region 20 by pressing on the transparent, flexible panels. Interaction with the modeling compound 36 can include squishing the modeling compound 36 or moving the compound about the interior region 20. Modeling compounds of different colors can be arranged in the interior region 20. Aesthetic designs or images can be created with the modeling compound 36, or modeling compounds of different colors, and inserted into the stacker 12 for display through the transparent panel. Interaction with the modeling compounds can include mixing the modeling compounds. Modeling compounds for use in the system 10 of the disclosure can be, for example, thermochromic or photochromic. Thermochromic modeling compounds can be adapted to change color when the modeling compound is interacted with through the transparent panel through heat generated by the user pressing on the modeling compound through the transparent panel. Photochromic compounds can change color upon exposure to light through the transparent panel. The system 10 can further include a light source for use to active the photochromic properties of a modeling compound 36 through the transparent panel.

The system 10 can also be used for displaying the modeling compound. For example, the modeling compound can be shaped into a desire shaped and placed between the walls 14 such that the shape is visible through the transparent panel or transparent panels 18. Multiple stackers 12 can be arranged in various structures, such as a house, and modeling compound shapes can be displayed, for example, as windows, such as shown in FIG. 6.

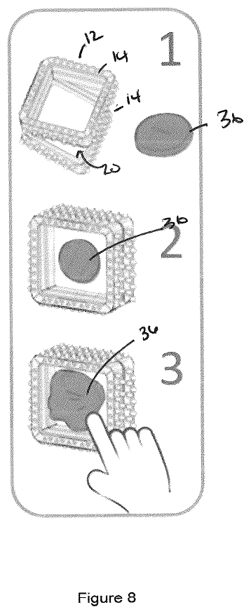

Systems 10 in accordance with the disclosure can include stackers 12 having a plurality of difference shapes. Any shape can be used. For example, block-like shapes can be provided in the system 10 to build structures. The system 10 can include shapes that can be assembled to define an animal shape, for example (FIG. 7). The interior region 20 can be used to incorporate a modeling compound to give color, facial features, or other characteristics to the shaped stackers 12. Any shapes and combinations of shapes for the stackers 12 can be included in the system 10.

The system 10 can include any modeling compound. For example, a modeling compound such as described in U.S. Pat. No. 8,633,269, can be used with the stackers 12.

What is claimed is:

1. A sensory play system, comprising:
one or more stackers, each stacker comprises two walls connected at a hinge and designed to be moved between open and closed positions, one or both of the two walls comprising a transparent panel, wherein in the closed position, an interior region is defined between the two walls; and
a modeling compound to be received in the interior region.

2. The system of claim 1, further comprising a modeling compound, wherein the modeling compound can be received in the interior region and interacted with by a user through the transparent panel on one or both of the two walls.

3. The system of claim 2, wherein the transparent panel is flexible.

4. The system of claim 1, wherein each of the two walls comprise a transparent panel, the transparent panel of each wall being aligned with one another.

5. The system of claim 1, wherein the stacker further comprises a plurality of pins extending outwardly from at least a portion of the perimeter of at least one the two walls.

6. The system of claim 1, comprising a first stacker having at least one pin extending outwardly from a portion of the perimeter of at least one of the two walls and a second stacker having an aperture in a perimeter of at least one of the two walls, the aperture sized to receive the pin to thereby connect the first and second stackers 12.

7. A sensory play system comprising:
two or more stackers, each stacker comprises two walls connected at a hinge and designed to be moved between open and closed positions, one or both of the two walls comprising a transparent panel, wherein in the closed position an interior region is defined between the two walls; and
a modeling compound to be received in the interior region, wherein:
each of the two or more stackers 12 comprise one or more pins extending outwardly form at least a portion of the perimeter of at least one wall;
the pins of each of the two or more stackers 12 are adapted to engage with pins and/or apertures formed between pins of another one of the two or more stackers 12 to thereby connect stackers 12 to one another.

8. The sensory play system of claim 7, wherein at least one of the two or more stackers 12 comprises the one or more pins arranged in two or more rows, wherein a space between the two or more rows is adapted to receive one or more pins from another one of the two or more stackers 12 to thereby connect the stackers 12 to one another.

9. The sensory play system of claim 8, wherein the one or more pins are arranged in three or more rows.

10. The sensory play system of claim 7, wherein each of the two or more stackers 12 comprises one or more pins extending outwardly from all edges of the perimeter of each of the walls of the stacker.

11. The sensory play system of claim 7, wherein at least one of the two or more stackers is in the shape of a triangle, a square, a half circle, or a rectangle.

12. The system of claim 7, wherein the transparent panel is flexible.

13. The system of claim 7, wherein each of the two walls comprise a transparent panel, the transparent panel of each wall being aligned with one another.
